(19) United States
(12) Patent Application Publication
(10) Pub. No.: US 2010/0303932 A1
(43) Pub. Date: Dec. 2, 2010

(54) Method for Attenuating the Effects of Botulinum Toxin

(76) Inventors: Baskaran Thyagarajan, Scotch Plains, NJ (US); Joseph J. McArdle, Annandale, NJ (US)

Correspondence Address: Jane Massey Licata;Licata & Tyrrell P.C., 66 E. Main Street, Marlton, NJ, 08053, US

(21) Appl. No.: 12/788,575

(22) Filed: May 27, 2010

Related U.S. Application Data
(60) Provisional application No. 61/217,341, filed on May 28, 2009.

(51) Int. Cl. A61K 33/00; A61K 31/165; A61P 39/00

(52) U.S. Cl. 424/722; 514/627

(57) ABSTRACT

The present invention is a method for attenuating the effects of botulinum toxin poisoning using capsaicin and/or a capsaicin analog.

### INTRODUCTION

This application claims benefit of priority to U.S. Provisional Application Ser. No. 61/217,341, filed May 28, 2009, the content of which is incorporated herein by reference in its entirety.

This invention was made with government support under contract number RO1 NS 045979 awarded by National Institutes of Health. The government has certain rights in the invention.

### BACKGROUND OF THE INVENTION

Neurotoxins produced by the anaerobic bacterium Clostridium botulinum are the most potent biological toxins. Botulinum neurotoxin A (BoNT/A) is the most toxic naturally occurring protein. BoNT/A cleaves SNAP-25 and inhibits acetylcholine release from motor nerve endings which leads to paralysis of skeletal muscles. Although BoNTs are potential weapons for bioterrorism (Amon, et al. (2001) JAMA 285:1059-1070), they are increasingly used for the treatment of a variety of neurological and cardiovascular diseases, as well as for cosmetic purposes (Schnider, et al. (1994) Wien Klin. Wochenschr. 106:335-344; Benedetto (1999) Int. J. Dermatol. 38:641-655; Tsuboi, et al. (2002) Jpn. J. Pharmacol. 89:249-254; Scott (2004) Dermatol. Clin. 22:131-133; Bilici, et al. (2007) AJR Am. J. Roentgenol. 189:W143-W145; Van Beek, et al. (2007) Plast. Reconstr. Surg. 119:217-226; Schulte-Mattler (2008) CNS Drugs 22:725-738). These clinical uses provide a potential danger of accidental intoxication.

The seven serotypes of neurotoxin, designated BoNT/A to G (Oguma, et al. (1995) Microbiol. Immunol. 39:161-168), are each composed of a heavy (HC) and light (LC) chain linked by a disulfide bond. The primary toxic effect is inhibition of vesicular acetylcholine (ACh) release from motor nerve terminals, which leads to muscle paralysis. To achieve this effect, BoNTs undergo a cascade of cellular events (Simpson (1981) Pharmacol. Rev. 33:155-188), which begin with HC binding to specific extracellular receptors followed by holotoxin endocytosis into the nerve terminal (Yowler, et al. (2002) J. Biol. Chem. 277:32815-32819; Dong, et al. (2006) Science 312:592-596). The HC forms a pore in the endosomal membrane which enables translocation of the LC zinc endoprotease into the cytosol (Montal, et al. (1992) FEBS Lett. 313:12-18; Brunger, et al. (2007) PLoS Pathog. 3:1191-1194). The LC chain cleaves specific SNARE (Soluble Nethylmaleimide-sensitive factor Attachment REceptor complex) proteins which are essential to exocytosis of Ach-enriched vesicles. BoNT/A LC cleaves synapse associated protein of 25,000 Daltons (SNAP-25; (Blasi, et al. (1993) Nature 365:160-163)) and is the most potent and long acting BoNT serotype.

Active and passive immunization have been used to protect against and treat from poisoning with BoNTs. However, the increasing applicability of BoNTs to treat human diseases makes active immunization less desirable. At the same time, antitoxin is most effective during the early phase of poisoning with BoNTs. Once BoNT enters the intracellular space, antibodies would not have access to the toxin. In this situation, severely poisoned patients depend upon artificial ventilation for long periods of time (Souayah, et al. (2006) Neurology 67:1855-1856). Therefore, it is important to develop effective prophylactic and therapeutic measures against BoNT/A.

### SUMMARY OF THE INVENTION

The present invention features a method for attenuating the effects of botulinum toxin poisoning by administering to a subject in need thereof an effective amount of capsaicin or capsaicin analog thereby attenuating the effects of botulinum toxin poisoning. In some embodiments the botulinum toxin is botulinum toxin A or B. In other embodiments, the capsaicin or capsaicin analog is administered prior to or after exposure to botulinum toxin. In certain embodiments, the capsaicin or capsaicin analog is administered in combination with calcium and may be administered orally, intraperitoneally, intravenously, or intramuscularly.

### DETAILED DESCRIPTION OF THE INVENTION

It has now been found that mammalian motor nerve terminals express TRPV1, the receptor for capsaicin and that in vivo and in vitro treatment with capsaicin reduces the effects of BoNT/A on motor nerve endings. This action is attributed to inhibition of BoNT/A uptake by capsaicin.

Interactions between capsaicin and clostridial toxins have been demonstrated (Mantyh, et al. (1996) Gastroenterology 111:1272-1280; Meng, et al. (2007) J. Cell Sci. 120:2864-2874). Capsaicin is an agonist of the transient receptor potential channel, TRPV1. In the present study, western blot analysis demonstrated that adult mouse motor nerve terminal endogenously expresses the capsaicin receptor, TRPV1. Furthermore, immunocytochemistry revealed TRPV1 on motor nerve endings. In agreement with other investigators (Lakshmi & Joshi (2005) Cell Mol. Neurobiol. 25:819-832), the instant data also demonstrated expression of TRPV1 on cholinergic Neuro 2a cells. Moreover, this study clearly demonstrated that capsaicin protects function of the neuromuscular junction against BoNT/A. It has been demonstrated that BoNT/A reduces FM1-43 uptake into nerve endings of the mouse diaphragm muscle (Henkel, et al. (1996) Proc. Natl. Acad. Sci. USA 93:1918-1923), whereas capsaicin pretreatment preserved exocytosis-induced FM1-43 uptake into cholinergic neurons thereby protecting their exocytotic mechanism from BoNT/A. In so far as cleavage of SNAP-25 by botulinum serotype A fulfills the requirements of the multistep model of botulinum toxin action that includes receptor-mediated endocytosis, pH-dependent translocation, and zinc-dependent proteolysis (Kalandakanond & Coffield (2001) J. Pharmacol. Exp. Ther. 296:980-986), it was contemplated that one mechanism of the prophylactic action of capsaicin is reduction of BoNT/A binding and/or uptake into cholinergic nerves. To demonstrate this action, uptake of fluorescently labeled BoNT/A into motor nerve terminals and Neuro 2a cells was examined. For both cell types, brief preincubation in capsaicin reduced uptake of BoNT/A, an effect that was prevented by removal of extracellular Ca²⁺. These results indicate that capsaicin protects mouse motor nerve endings from the neuroparalytic effects of BoNT/A by reducing toxin uptake. In addition to demonstrating prophylactic anti-BoNT/A action of capsaicin, recovery of CMAP amplitude after BoNT/A indicated that capsaicin also had a therapeutic effect, i.e., CMAP amplitude recovered more rapidly when capsaicin injection followed BoNT/A.

Accordingly, the present invention features a method for attenuating the effects of botulinum toxin poisoning by administering to a subject in need thereof an effective amount of capsaicin and/or capsaicin analog. There are seven serotypes of BoNTs, termed A, B, Cl, D, E, F, G (Hathaway (1990) Clin. Microbiol. Rev. 3:66-98; Oguma, et al. (1995) Microbiol. Immunol. 39:161-168). The existence of two classes of binding sites distinguished by different affinities and protease sensitivities has led to dual-receptor concept: complex gangliosides first accumulate clostridial neurotoxins on the plasma membrane before protein receptors subsequently mediate their endocytosis. In so far as it has been demonstrated that trisialoganglioside GT1b interacts with the receptor binding domains of BoNT/A and BoNT/B, certain embodiments of the invention feature attenuating the effects of BoNT/A and BoNT/B, with particular embodiments embracing the mitigation of BoNT/A.

As used herein, the term “capsaicin” refers to trans 8-methyl-N-vanillyl-6-noneamide. Analogs of capsaicin with similar physiological properties are known in the art and include, e.g., homocapsaicin, dihydrocapsaicin, nordihydrocapsaicin, homodihydro-capsaicin, and norcapsaicin. Furthermore, reinsiferatoxin is described as a capsaicin analog in U.S. Pat. No. 5,290,816. U.S. Pat. No. 4,812,446 describes other capsaicin analogs and methods for their preparation. Furthermore, Ton, et al. ((1995) Brit. J. Pharm. 10:175-182) discuss the pharmacological actions of capsaicin and its analogs. Where a capsaicin analog is selected to replace some or all of the capsaicin, the analog can be selected from those analogs with similar physiological properties to capsaicin as are known in the art.

Useful capsaicin compositions can be obtained by preparing capsaicin and/or analogs (e.g., by chemical synthesis or extraction from a natural source), isolating capsaicin and/or analogs to near homogeneity (e.g., at least 95%), and mixing the capsaicin and/or capsaicin analogs to a desired concentration by weight, in a pharmaceutically acceptable carrier for oral, intraperitoneal, intravenous, or intramuscular administration for example. Such carriers are well-known to those of skill in the art and can vary depending on the means of administration.

For example, pharmaceutical compositions suitable for injectable use include sterile aqueous solutions or dispersions and sterile powders for the extemporaneous preparation of sterile injectable solutions or dispersion. Generally, the composition is sterile and is fluid to the extent that easy syringability exists. It is typically stable under the conditions of manufacture and storage and is preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), or suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the capsaicin and/or capsaicin analog in the required amount in an appropriate solvent with one or a combination of ingredients enumerated above, as required, followed by filtered sterilization. Generally, dispersions are prepared by incorporating the capsaicin and/or capsaicin analog into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The capsaicin and/or capsaicin analog and other ingredients may also be enclosed in a hard or soft shell gelatin capsule, compressed into tablets, or incorporated directly into the subject's diet. For oral therapeutic administration, the capsaicin and/or capsaicin analog may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the capsaicin and/or capsaicin analog in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of capsaicin and/or capsaicin analog calculated to produce the desired therapeutic effect in association with the required pharmaceutical carrier. The specification for the dosage unit forms of the invention may be dictated by and directly dependent on the characteristics of the capsaicin and/or capsaicin analog and the particular therapeutic effect to be achieved, and the limitations inherent in the art of compounding such a therapeutic compound for the treatment of a selected condition in a subject.

As indicated, the capsaicin and/or capsaicin analog of the invention are of use in attenuating the effects of botulinum and are therefore of use in the treatment and prevention of botulinum toxin poisoning. In this respect, the term “attenuating” refers to a reduction, prevention, mitigation, elimination or substantial diminution of one or more symptoms characteristic of botulinum toxin poisoning, e.g., ptosis, gaze paralysis, descending paralysis, weak or nasal voice, poorly reactive or dilated pupils, nystagmus, diminished gag reflex, tongue weakness, dry mouth, and pharyngeal erythema. “Treatment” or “treating” includes (1) inhibiting botulism in a subject or patient experiencing or displaying the well-known symptomatology of botulism, (2) ameliorating botulism in a subject or patient experiencing or displaying the well-known symptomatology of botulism, and/or (3) effecting any measurable decrease in botulism symptomatology. “Prevention” or “preventing” includes: (1) inhibiting the onset of botulism in a subject who may be at risk and/or predisposed to botulinum toxin poisoning (e.g., upon eating food suspected of being contaminated with botulinum toxin or Clostridium botulinum bacteria) but does not yet experience or display any or all of the pathology or symptomatology of the disease, and/or (2) slowing the onset of the pathology or symptomatology of botulism in a subject who may be at risk and/or predisposed to botulinum toxin poisoning but does not yet experience or display any or all of the pathology or symptomatology of the disease.

Capsaicin and/or one or more capsaicin analogs are administered at a therapeutically effective dosage sufficient to attenuate botulinum toxin poisoning. As used herein, a subject in need of treatment refers to a living mammalian organism, such as a human, monkey, cow, dogs, cat, or horses that has been exposed or suspected of being exposed to botulinum toxin or Clostridium botulinum bacteria. In certain embodiments, the subject is a primate. Non-limiting examples of human subjects are adults, juveniles, and infants. The term “effective,” as that term is used in the specification and/or claims, means adequate to accomplish a desired, expected, or intended result of reducing, preventing, or eliminating one or more sings or symptoms characteristic of botulinum toxin poisoning.

As demonstrated herein, the protective effect of capsaicin was dependent upon the presence of calcium. Accordingly, one embodiment of the invention further includes administering the capsaicin and/or capsaicin analog in combination with calcium. Moreover, the capsaicin and/or capsaicin analogs can be used alone in the mitigation of botulinum toxin poisoning or in combination with other known treatments including, e.g., Trivalent Botulinum Antitoxin or Heptavalent Botulinum Antitoxin.

The actual dosage amount of capsaicin and/or capsaicin analog, and optional calcium, or composition comprising capsaicin and/or capsaicin analog, and optional calcium, administered to a subject may be determined by physical and physiological factors such as age, sex, body weight, severity of condition, previous or concurrent therapeutic interventions, and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

The following non-limiting examples are provided to further illustrate the present invention.

### Example 1

### Material and Methods

Animals. All animal procedures were approved by the Institutional Animal Care and Use Committee. Adult Swiss Webster mice were anesthetized with an intraperitoneally injected mixture of ketamine (100 mg/kg) and xylazine (9 mg/kg) prior to surgical exposure of the peroneal nerve's entry into the EDL muscle. Three μl of 6.67 μM BoNT/A in HEPES buffer was injected bilaterally into the space surrounding the innervation site of the EDL with a 26 Gauge Hamilton syringe. The skin incision was then surgically closed with aseptic technique. In experiments with capsaicin, the drug was either bilaterally coinjected (3 μl of 1 mM stock solution) with BoNT/A or injected 4 or 8 hours prior to BoNT/A. To test for a prophylactic affect of capsaicin on body weight and muscle performance, animals were injected in both hind limbs with either BoNT/A alone or with capsaicin followed by BoNT/A. To test the therapeutic effects of capsaicin, mice were injected with capsaicin 12 hours after injection of BoNT/A. This time was selected to ensure that mice showed signs of inhibition of toe spread reflex before capsaicin injection.

Cell Culture. Mouse cholinergic neuroblastoma (Neuro 2a) cells were cultured in DMEM-F12 medium (pH 7.4) supplemented with 10% Fetal Bovine Serum and antibiotics. Two groups of Neuro 2a cells were seeded on poly-L-lysine-coated cover slips. The experimental group was incubated (room temperature) in HEPES Ringer solution containing 10 μM capsaicin for 15 minutes and subsequently washed. Both groups were then incubated in 10 μM BoNT/A in 40 mM KCl containing HEPES Ringer solution for 30 minutes to induce neurotoxin uptake. A portion of the capsaicin-free group was treated with 40 mM KCl in HEPES Ringer alone as control. Following wash with HEPES, the preceding three groups of Neuro 2a cells were incubated for 10 minutes in 40 mM KCl HEPES containing FM 1-43 (1 μg/ml). Next, these three groups of Neuro 2a cells were washed twice with HEPES buffer and observed with a ZEISS (Oberkochen, Germany) LSM-510 confocal microscope, equipped with an argon laser (488 nm). Images were saved as TIFF files and analyzed with ImageJ software.

Immunoblotting, Confocal Microscopy, and Immunocytochemistry. EDL, diaphragm, or Triangularis sterni (McArdle, et al. (1981) J. Neurosci. Methods 4:109-115) nerve muscle preparations were dissected from isoflurane anesthetized Swiss Webster or yellow fluorescence protein (YFP) expressing mice. Harvested diaphragm preparations were flash frozen in liquid nitrogen and stored at −80° C. until tissue was homogenized in phosphate-buffered saline (PBS) containing 1% NP-40 and complete protease inhibitor cocktail (Amersham, USA). Homogenates of Neuro 2a cells were prepared in the same manner. After centrifuging the homogenate at 14,000 rpm for 10 minutes in the cold, the crude lysate (40 μg) was resolved via SDS-PAGE after boiling in 1× Laemmli buffer. Immunoblotting was performed with anti-rabbit TRPV1 antibody (Santa Cruz Biotechnology, Inc., USA).

Triangularis sterni nerve-muscle preparations or phrenic nerve diaphragm preparations were pinned to a Sylgard-lined PLEXIGLAS chamber and bathed in HEPES Ringer solution. For evaluation of neurotoxin uptake, diaphragm preparations of YFP mice were bathed in 667 pM ALEXA 647-labeled BoNT/A and toxin uptake initiated in response to nerve stimulation (1 Hz) or exposure to HEPES Ringer containing 40 mM KCl (osmolarity adjusted by reducing NaCl to 100 mM) as well as 667 pM ALEXA 647 BoNT/A. For immunocytochemistry, nerve-muscle preparations were fixed in 4% formaldehyde PBS (room temperature) for 1 hour followed by overnight incubation (4° C.) in 100 mM glycine in PBS. Preparations were then permeabilized with 1% TRITON X-100 in PBS at 4° C. for 6 hours followed by three washes with PBS and blocking with 2% BSA in PBS over night. Postsynaptic ACh receptors were then labeled by exposing the fixed tissue to 1 ng/ml of ALEXA 488-labeled α-bungarotoxin (α-BnTX) at 4° C. for 6 hours. The muscle endplate region was cut out and mounted in VECTASHIELD on a slide and kept frozen at −20° C. prior to imaging with a NIKON LSM-410 confocal microscope equipped with argon (488 nm for α-BTX) and HeNe (647 nm for BoNT/A) lasers. Images were saved and represented as TIFF files.

For the immunohistochemical detection of TRPV1 in hemidiaphragm preparations, freshly isolated tissues were fixed as described above and labeled with anti-rabbit TRPV1 antibody (Santa Cruz Biotechnology, Inc. USA; 1:200 dilution in PBS). The preparations were then washed and stained with FITC-labeled secondary antibody (1:500 dilution in PBS) and subjected to confocal microscopy.

Neuro 2a cells grown on 25 mm round glass coverslips were fixed with 2 ml of 4% paraformaldehyde in PBS at room temperature for 30 minutes. The cells were washed twice with PBS (2 ml) and treated with 2 ml of 100 mM glycine in PBS for 30 minutes. After washing twice with PBS, the cells were permeabilized with 300 μl of cold methanol at −20° C. for 5 minutes and washed with 2 ml PBS. The cells were then blocked with 2% BSA for 1 hour at 37° C. followed by a single wash with PBS and incubation for another 1 hour in 300 μl of anti-rabbit TRPV1 antibody in PBS (1:200 dilution). Next, the cells were washed once with PBS, incubated for 1 hour in the presence of FITC-labeled secondary antibody (1:500 dilution in PBS), washed three times with 2 ml PBS, washed once with distilled water, and mounted with VECTASHIELD on a glass slide. The slides were stored at −20° C. until cells were examined with a ZEISS (Oberkochen, Germany) LSM-510 confocal microscope equipped with an argon laser (488 nm). Images were saved as TIFF files.

In vivo Study of Muscle Performance. To evaluate motor strength and coordination, mice were first allowed to adapt to a rotating rod (Rotamex-5, Columbus Instruments, Inc). After 1 week of training, control mice were capable of 300 seconds of sustained performance on a rod rotating at 15 RPMs. Rotarod performance was tested biweekly following administration of BoNT/A alone or in combination with capsaicin. Mean duration of time on the rotarod was averaged for three repeated runs separated by 30-minute intervals. For each run, mice were kept on the apparatus for no longer than 300 seconds. Mice were tested in groups of four at each time point after BoNT/A. Tests were performed in mice that were treated with capsaicin 12 hours after BoNT/A exposure to evaluate the therapeutic effects of capsaicin.

The toe spread reflex was scored from 1 to 5 depending on the number of toes which the mouse could extend when lifted by the tail. The leg strength was scored by evaluating the ability of the mouse to move the legs and grip a rod: 0, no movement toward rod; 1, moves slightly toward rod but profound weakness prevents gripping the rod to support body weight; 2, supports body weight on the rod with a weak grip; 3, can hold onto rod for at least 30 seconds; 4, easily reaches for and grips rod to support body weight for at least 1-2 minutes; 5, quickly grasps rod with fast and strong grip which supports body weight for 2 or more minutes. The waist muscle strength was evaluated and scored based on the ability of the mouse to show movement of the waist when held by its tail: 0, no movement; 1, very slight movement; 2, attempts to lift itself onto a rod; 3, can easily reach for, lifts itself onto, and climb over a suspended rod; 4, reaches for a rod and holds onto it with greater strength; 5, can reach to the rod easily, hold onto it, lifts itself onto the rod with ease. Groups of four mice were scored biweekly and the scores obtained for individual animals in each group were pooled and averaged.

For electromyographic analysis, mice were anesthetized with an ip injection of ketamine (100 mg/kg) and xylazine (9 mg/kg). After shaving their abdomen and distal hind limbs, mice were taped prone to a polystyrene foam board. A heated pad maintained body temperature between 32 and 38° C. Stimulating electrodes were 0.7 mm needles insulated with POLYTEF (Dantec sensory needle, Skorlunde, Denmark); the cathode was placed close to the sciatic nerve in the proximal thigh and the anode was placed subcutaneously 1 cm proximally. Motor responses of flexor and extensor muscles were recorded with a ring electrode placed around the animal's hind limb, distal to the site of BoNT/A injection. The reference electrode was a ring electrode placed 1.2 cm distal to the recording electrode. Distance between stimulating and recording electrodes was 1.5 to 2 cm. Both the right and left hind limbs were studied. A ground electrode was placed in the contralateral limb. Muscle nerves were stimulated with monophasic pulses of constant current delivered through a constant current stimulator with fine intensity control (AM 2100). Recordings were made through Dantec motor amplifiers (KEYPOINT) connected to a computer for digital storage and off-line analyses. Filter settings were 500 Hz/5 KHz. A threshold of less than 0.7 mA for evoking a motor response indicated that the stimulating electrode was optimally positioned for nerve stimulation. Stimulus intensity was increased until the compound muscle action potential (CMAP) amplitude reached maximum. CMAP amplitude (peak-peak) and distal latency (to negative onset) were then recorded. Recordings were made for both hind limbs and the averaged results were computed. EMG analysis was made a maximum of two times for an individual mouse.

In vitro Evaluation of Muscle Performance and Electrophysiology of the Neuromuscular Junction. EDL preparations from BoNT/A alone treated or capsaicin pretreated mice were dissected and mounted in a glass chamber (Rodnoti Glass Technology, Inc., Monrovia, Calif.) filled with oxygenated (95% O₂-5% CO₂) normal Ringer solution (pH 7.4, 37° C.) containing (mM) NaCl (135), KCl (5), MgCl₂ (1), CaCl₂ (2), Na₂HPO₄ (1), NaHCO₃ (15), glucose (5.5). The EDL nerve was drawn into a suction electrode for indirect activation of muscle twitches. One tendon of the muscle was tied to a Grass Force transducer connected to an Axon Instruments (Molecular Devices, Sunnyvale, Calif.) DIGIDATA 1440A. This enabled acquisition and analysis with PCLAMP software of muscle mechanical responses to nerve stimulation. Isolated preparations were adjusted to optimal length for force generation and equilibrated for 15 minutes prior to stimulation at 0.1 Hz for data acquisition.

EDL preparations were dissected from control, capsaicin alone, BoNT/A alone, BoNT/A after capsaicin pretreated mice, pinned to a SYLGARD-lined PLEXIGLASS chamber, and bathed in HEPES-buffered physiological solution. In order to activate transmitter release, the muscle nerve was drawn into a suction electrode and stimulated supramaximally. Conventional current clamp or two electrode voltage clamp (AXOCLAMP 2 B, Molecular Devices, Sunnyvale, Calif.) allowed evaluation of stimulus evoked endplate potentials (EPPs) or currents (EPCs; −75 mV holding potential) at 1, 20, 50, and 70 Hz. For control preparations, 0.75 μM Conotoxin GIIIB was added to the physiologic solution to prevent mechanical responses to nerve stimulation.

Chemicals and Drugs. BoNT/A was obtained from Metabiologics Inc., WI. Labeled toxins were obtained from BB Tech (MA). All other chemicals and drugs were obtained from Sigma (USA). μ Conotoxin GIIIB and α-Bungarotoxin (α-BnTX) were obtained from Alamone Labs (Israel) and Invitrogen (USA), respectively.

Data Analyses. Data for all figures were expressed as mean±S.E.M. Student's t test evaluated the statistical significance of population mean differences with ** and *** indicating p<0.001 and <0.0001, respectively.

### Example 2

### Prophylactic Anti-BoNT/A Action of Capsaicin

The search for antidotes to BoNTs is complicated by the multistep process whereby these proteins reach their primary substrate within motor nerve endings. This complexity undoubtedly contributes to the “disconnect” (Eubanks, et al. (2007) Proc. Natl. Acad. Sci. USA 104(8):2602-7) between in vitro and in vivo observations, which confounds the search for useful BoNT/A antidotes. That is, development of agents that target an apparently well-understood step in BoNT/A action in a simplified system may mislead research aimed at antidote development. To minimize this disconnect, in vivo and in vitro experiments were focused on the cholinergic motor nerve ending, which is the primary target of BoNT/A.

To determine the effects of capsaicin on BoNT/A, capsaicin was injected into the right hind limb of mice 4 hours prior to bilateral injection of BoNT/A. Toe spread reflex (TSR) scores were obtained from mice that were injected with BoNT/A either 5-10 minutes before BoNT/A (n=18) or 4 (n=10) or 8 (n=4) hours after an injection of capsaicin into the same hind limbs. While BoNT/A abolished the TSR in all of the control mice (n=36), this reflex remained completely normal for each of the three capsaicin treated groups. Within 24 hours, the toe spread reflex (TSR) was totally abolished in the left limb, while the reflex remained normal in the capsaicin pre-injected right limb. At times later than 24 hours after the BoNT/A injection, TSR continued unaltered for all of the capsaicin pretreated mice.

To test in vivo muscle function and coordination after BoNT/A, the time mice could walk on a rod rotating at 15 RPM was measured. Maximal duration of “walk time” was set to 300 seconds since all non-treated control mice performed for this duration. Beginning at 24 hours after BoNT/A injection, walk time significantly declined below control and reached a minimal value of 127±48 seconds at 4 days after toxin. The walk time gradually recovered to the control duration at about 20 days after BoNT/A. In contrast, mice injected with capsaicin at 4 hours prior to BoNT/A remained equivalent to non-treated controls in walk time. Capsaicin pre-injection also reduced the loss of body mass (Table 1) and cessation of grooming behavior associated with the preceding BoNT/A-induced functional abnormalities.

TABLE 1

| | Body Weight (gram) ± SEM | | |
|---|---|---|---|
| | Week 2 | Week 4 | Week 6 |
| Control | 30.39 ± 0.44 | 33.44 ± 0.37 | 39.92 ± 0.88 |
| +BoNT/A | 32.66 ± 0.38 | 24.02 ± 0.37 | 33.02 ± 0.12 |
| +Capsaicin | 33.08 ± 0.48 | 31.97 ± 0.53 | 38.44 ± 0.78 |
| +BoNT/A | | | |
| +Capsaicin | 32.62 ± 0.56 | 33.55 ± 0.51 | 38.39 ± 0.93 |

*Values are mean ± SEM for 12 to 32 mice.

Furthermore, assessment of muscle behavior by an investigator blinded to the mouse treatment indicated that capsaicin significantly protected muscle strength and coordination against BoNT/A. That is, motor strength score was significantly less than control at 2 and 3 weeks after BoNT/A alone. In contrast, capsaicin pre-injected mice retained normal motor strength at all times after BoNT/A (Table 2). However, BoNT/A administered at times later than hours after capsaicin was fully active in suppressing the preceding indicators of neuromuscular function.

TABLE 2

| | Muscle Strength (%) ± SEM | | |
|---|---|---|---|
| | Week 2 | Week 4 | Week 6 |
| Control | 100 | 100 | 100 |
| +BoNT/A | 50.36 ± 3.40 | 70.02 ± 6.21 | 90.18 ± 3.68 |
| +Capsaicin | 98.04 ± 1.12 | 100 | 100 |
| +BoNT/A | | | |
| +Capsaicin | 100 | 100 | 100 |

*Values are mean ± SEM for 12 to 32 mice.

The preceding protective effects of capsaicin were associated with preservation of neuromuscular physiology in vivo as reflected by the compound muscle action potential (CMAP). While CMAP amplitude declined for both BoNT/A treated groups, the magnitude of the decline was significantly less for the capsaicin pre-injected mice. CMAP amplitude was greater at 4 and 8 weeks after BoNT/A for hind limbs pretreated with capsaicin as compared to the BoNT/A-alone treated mice. That is, CMAP amplitude was 11.3±2.3 (n=4), 0.7±0.28 (n=4), and 4.6±0.92 (n=4) mV at 4 weeks after BoNT/A for control, BoNT/A, and capsaicin pre-injected BoNT/A treated mice, respectively. At 8 weeks after BoNT/A, CMAP amplitude had partially recovered for the BoNT/A alone and capsaicin pre-injected groups to 2.6±0.79 (n=4) and 6.7±1.24 (n=4) mV, respectively.

At 24 hours after injection of capsaicin alone, mice exhibited normal muscle strength and body weight as well as CMAP amplitude and performance on the rotor rod.

To evaluate functional neuromuscular transmission, neurally evoked twitches were evaluated for EDL nerve muscle preparations isolated from mice injected with BoNT/A alone or at 4 hours after capsaicin. The results of this analysis indicated that the nerve-evoked muscle twitch tension was reduced at 48 hours after BoNT/A alone. Pre-injection of capsaicin significantly reduced this affect of BoNT/A, i.e., mean twitch tension was 140±60.0 (n=4) and 500±100 (n=4) mg for preparations that received BoNT/A alone and BoNT/A at 4 hours after capsaicin, respectively. Thus, while BoNT/A significantly reduced twitch tension after capsaicin, the reduction was less than for preparations treated with BoNT/A alone. In vivo administration of capsaicin alone had no effect on force generation or the amplitude of stimulus evoked endplate currents (EPC).

It was subsequently determined whether capsaicin pretreatment preserved neurotransmitter release, which in vivo treatment with BoNT/A alone inhibited. Based upon trains (70 Hz) of endplate potentials (EPPs) for EDL nerve muscle preparations isolated from control, BoNT/A alone, and BoNT/A after capsaicin treated mice, nerve stimulation failed to produce EPPs for BoNT/A treated preparations. In contrast, preparations treated with capsaicin prior to BoNT/A, produced EPPs which, similar to control, did not fail in response to the 70 Hz nerve stimulation.

Capsaicin also protected exocytosis-induced uptake of FM1-43 into Neuro 2a cells from the inhibitory effect of BoNT/A. Control Neuro 2a cells were uniformly labeled following a 10 minute incubation in 1 μg/ml FM1-43 in HEPES buffer containing 40 mM KCl. Neuro 2a cells preincubated in HEPES containing 10 μM BoNT/A and 40 mM KCl for 30 minutes were significantly less labeled when subsequently exposed to FM1-43. In contrast, FM1-43 labeling was equivalent to control for Neuro 2a cells pretreated with 10 μM capsaicin for 10 minutes prior to BoNT/A. Thus, capsaicin protects the exo- and endocytotic mechanisms of cholinergic Neuro 2a cells against BoNT/A. This protective effect of capsaicin on FM1-43 uptake in Neuro 2a cells did not occur in the absence of extracellular Ca²⁺.

Since capsaicin injection protected mice from the inhibitory effects of BoNT/A, expression of the capsaicin receptor, TRPV1 channel protein, was determined in mouse motor neurons. Western blot analysis indicated the presence of TRPV1 in HEK 293 cells transiently transfected with the cDNA for this protein, as well as for adult mouse diaphragm muscle and Neuro 2a cells. Immunocytochemical detection identified TRPV1 on a phrenic nerve ending innervating the diaphragm muscle of a mouse that expressed YFP in motor neurons. This antibody also detected TRPV1 on the surface of Neuro 2a cells.

If capsaicin protects against the neuroparalytic effects of BoNT/A via TRPV1 activation, then TRPV1 inhibition should antagonize the protective effects of capsaicin. To demonstrate this, capsazepine (TRPV1 antagonist; 3 μl of 1 mM) was injected prior to capsaicin (3 μl of 1 mM) and BoNT/A (3 μl of 6.67 pM). In capsazepine preinjected (15 minutes before capsaicin) mice, capsaicin failed to protect against the neuroparalytic effects of BoNT/A; capsazepine pretreated mice were unable to spread toes 24 hours post-capsaicin similar to mice injected with BoNT/A alone. Similar to EPPS, acute in vitro exposure to 10 pM BoNT/A dramatically reduced the amplitude of endplate currents (EPCs) of the isolated EDL nerve muscle preparation. However, pre-treatment with capsaicin reduced this action of BoNT/A, whereas when capsazepine was applied prior to capsaicin, BoNT/A reduced EPC amplitude to an amplitude equivalent to that observed after BoNT/A alone. As for capsaicin, capsazepine alone had no effect on EPC amplitude or time course.

The interaction between BoNT/A, capsaicin, and capsazepine in EDL nerve muscle preparations stimulated at frequencies of 20, 50, and 70 Hz was also determined. Forty nerve stimuli were applied at each frequency and the mean EPC amplitude was plotted against stimulus number. For each stimulus frequency, pretreatment with capsaicin reduced the inhibitory effect of BoNT/A. However, following capsazepine treatment the protective action of capsaicin was abolished. Table 3 summarizes the mean amplitude of EPC 1, 10, 20, 30, and 40 for, each train recorded for control, BoNT/A alone, BoNT/A after capsaicin, BoNT/A after capsaicin preceded by capsazepine.

TABLE 3

| | | | | |
|---|---|---|---|---|
| | Control | +BoNT/A | +CAP + BoNT/A | +CPZ + CAP + BoNT/A |
| Pulse | (n = 3, 9) | (n = 6, 12) | (n = 6, 12) | (n = 4, 12) |

20-Hz EPC amplitude (nA)

| | | | | |
|---|---|---|---|---|
| 1 | 170.69 ± 15.47 | 2.69 ± 8.23 | 100.23 ± 8.2 | 3.60 ± 2.52 |
| 10 | 156.86 ± 17.23 | 1.81 ± 3.45 | 113.45 ± 16.12 | 1.82 ± 3.52 |
| 20 | 157.51 ± 17.14 | 0.44 ± 2.28 | 105.52 ± 11.74 | 0.48 ± 2.14 |
| 30 | 163.41 ± 16.63 | 2.16 ± 3.06 | 109.45 ± 5.26 | 0.22 ± 3.14 |
| 40 | 160.61 ± 16.76 | 1.98 ± 3.26 | 107.21 ± 8.45 | 2.03 ± 3.19 |

50-Hz EPC amplitude (nA)

| | | | | |
|---|---|---|---|---|
| 1 | 188.44 ± 17.34 | 0.53 ± 3.24 | 136.53 ± 13.22 | 1.22 ± 1.45 |
| 10 | 199.08 ± 10.39 | 2.06 ± 2.14 | 148.23 ± 18.91 | 2.28 ± 1.43 |
| 20 | 175.21 ± 9.31 | 2.08 ± 2.18 | 135.84 ± 25.6 | 1.99 ± 0.91 |
| 30 | 173.42 ± 9.06 | 6.19 ± 2.43 | 128.99 ± 25.82 | 3.41 ± 1.39 |
| 40 | 173.18 ± 8.77 | 0.89 ± 1.56 | 109.92 ± 43.14 | 2.45 ± 1.46 |

70-Hz EPC amplitude (nA)

| | | | | |
|---|---|---|---|---|
| 1 | 229.37 ± 13.36 | 0.75 ± 1.33 | 149.34 ± 14.59 | 1.11 ± 2.41 |
| 10 | 248.28 ± 11.99 | 1.64 ± 0.85 | 237.08 ± 48.05 | 1.46 ± 1.52 |
| 20 | 243.03 ± 12.77 | 1.29 ± 1.78 | 130.29 ± 19.28 | 2.02 ± 1.31 |
| 30 | 247.28 ± 11.65 | 2.13 ± 1.18 | 121.19 ± 18.39 | 1.17 ± 1.24 |
| 40 | 246.31 ± 14.57 | 1.31 ± 0.54 | 122.63 ± 18.18 | 0.19 ± 1.46 |

Treatment of capsaicin or BoNT/A and capsaicin did not affect the expression of TRPV1 in isolated mouse hemidiaphragm nerve-muscle preparations. Preparations were isolated and treated with vehicle (control) or capsaicin (100 μM) or capsaicin followed by BoNT/A (10 pM) for 90 minutes. The treated preparations were then homogenized and probed for TRPV1 expression by western blotting. Neither capsaicin alone nor treatment with capsaicin +BoNT/A affected the expression of TRPV1.

Studies with ALEXA 647 BoNT/A indicated that capsaicin inhibited toxin uptake into cholinergic nerves. Confocal images of motor endplates in Triangularis sterni nerve muscle preparations labeled with ALEXA 488 α-BnTX were analyzed, as were the corresponding motor nerve endings labeled with ALEXA 647 BoNT/A whose uptake was initiated in response to 1.5 hours of 1 Hz nerve stimulation (n=6) or incubation in 40 mM KCl (n=6). These images indicated that a 30-minute preincubation in 100 μM capsaicin prevented exocytosis-induced uptake of BoNT/A into motor nerve terminals. Similarly, capsaicin pretreatment reduced BoNT/A uptake into Neuro 2a cells depolarized with 40 mM KCl.

### Example 3

### Therapeutic Anti-BoNT/A Action of Capsaicin

The preceding results indicated that capsaicin exerts a prophylactic effect against BoNT/A. To test for a therapeutic action, capsaicin was injected into mice at 12 hours after BoNT/A. The 12-hour time point was selected since the toe spread reflex was qualitatively reduced. The following four groups of mice were tested: control, capsaicin alone injected, BoNT/A alone injected, and BoNT/A followed at 12 hours with capsaicin (n=4-8 for each group). The results' of this analysis indicated that capsaicin treatment 12 hours post BoNT/A exposure significantly enhanced recovery of body weight following BoNT/A. While CMAP amplitude declined for both BoNT/A alone as well as capsaicin post BoNT/A groups, the latter showed an enhanced rate of recovery. This was observed as a greater CMAP amplitude at 3 and 7 weeks after BoNT/A. That is, capsaicin treated groups showed better recovery from the BoNT/A mediated inhibition of CMAP. Moreover, CMAP amplitude was significantly greater at 3 and 7 weeks after BoNT/A for hind limbs that received capsaicin injection as compared to the BoNT/A alone treated mice. CMAP amplitude at week 3 after capsaicin alone (7.6±1.12 mV; n=4) was equivalent to control (7.8±1.18 mV). When comparing the fold recovery of CMAP from week 1 to week 7 for mice that received BoNT/A alone or capsaicin at 12 hours after BoNT/A, the former exhibited an approximately 7.5-fold recovery, whereas the latter exhibited an approximately 18-fold recovery. These results indicate that capsaicin treatment post-BoNT/A exposure significantly enhances the recovery from BoNT/A-induced neuroparalytic effects.

What is claimed is:

1. A method for attenuating the effects of botulinum toxin poisoning comprising administering to a subject in need thereof an effective amount of capsaicin or capsaicin analog thereby attenuating the effects of botulinum toxin poisoning.

2. The method of claim 1, wherein the botulinum toxin is botulinum toxin A or B.

3. The method of claim 1, wherein the capsaicin or capsaicin analog is administered prior to exposure to botulinum toxin.

4. The method of claim 1, wherein the capsaicin or capsaicin analog is administered after exposure to botulinum toxin.

5. The method of claim 1, further comprising administering the capsaicin or capsaicin analog in combination with calcium.

6. The method of claim 1, wherein the capsaicin or capsaicin analog is administered orally, intraperitoneally, intravenously, or intramuscularly.
